(19) United States
(12) Patent Application Publication
(10) Pub. No.: US 2022/0338872 A1
(43) Pub. Date: Oct. 27, 2022

(54) A SURGICAL SHARPS STORAGE SYSTEM

(71) Applicant: Averard Medical Devices, Galway (IE)

(72) Inventors: Jack Kelly, Galway (IE); Therese Healy Kelly, Galway (IE)

(21) Appl. No.: 17/765,957

(22) PCT Filed: Oct. 5, 2020
(86) PCT No.: PCT/EP2020/077791

§ 371 Date: Apr. 1, 2022

(30) Foreign Application Priority Data
Oct. 4, 2019 (IE) S2019/0165

(51) Int. Cl. A61B 17/06

(52) U.S. Cl. CPC A61B 17/06061; A61B 17/06114; A61B 17/06161; A61B 2017/06142

(57) ABSTRACT

The present invention provides a storage system for so called “sharps”, in particular but not limited to needles, used in surgical and medical procedures and comprising a body defining a first storage area for one or more packaged sharps; a second storage area for one or more empty sharps packages; and a third storage area for securing one or more used sharps.

### FIELD OF THE INVENTION

The present invention is concerned with a storage system for so called “sharps”, in particular but not limited to needles, used in surgical and medical procedures and provides a simple, inexpensive yet highly effective system for storing and tracking the use of such sharps, preferably before and after use.

### BACKGROUND OF THE INVENTION

Surgical sharps is the term given to any surgical or medical equipment including one or more sharp edges, points or similar surfaces or features capable of piercing or cutting, and which is generally understood to mean suture needles, scalpel blades, syringe needles, etc.

Disposal of surgical sharps following use is extremely important in the surgical and medical setting, both to avoid unintentional injuries to medical staff and/or the patient, and to account for all such needles or the like that have been used during the course of a procedure. For example where one or generally more suture needles are used in a surgical procedure, the individual needles are each provided in a sterile packaging, which once opened is retained but is generally simply discarded on a table or the like, and will often be inadvertently lost, for example falling on to the floor of the theatre.

The needles, once used are also retained and will conventionally be placed on an adhesive pad or attached to a magnetic strip. In addition, they are often placed in polyurethane foam. There is no device available that allows the unopened packs, opened packs and used or unused needles to be retained in the same device, facilitating a correct count during and after surgery. Once the used needles have been correctly counted at the end of the procedure, they are matched with the number of empty suture packets, which as mentioned are usually thrown around the table. If the empty suture packets have fallen on the floor or are otherwise misplaced, the count is incorrect. This may result in the patient being unnecessarily x-rayed in a search for missing needles. In addition, the needles are not matched with the packages with any semblance of organisation.

A number of collection devices currently exist to catch the used needles and to place them in a single area. One example of such a device includes a polyurethane foam block into which the used needles may be inserted for retention.

It is an object of the present invention to provide an improved means of collecting and collating used medical sharps and the packaging thereof, and which allows safe handling of sharps both when being opened for use and collected following use, and which is preferably a simple design embodying ease of use, and having low cost and minimal disposal volume.

### SUMMARY OF THE INVENTION

According to a first aspect of the present invention there is provided a surgical sharps storage system comprising a body defining a first storage area for one or more packaged sharps; a second storage area for one or more empty sharps packages; and a third storage area for securing one or more used sharps.

Preferably, the body is formed from at least one planar blank comprising a plurality of panels foldable to form at least one of the first, second and/or third storage areas.

Preferably, the first storage area comprises at least one receptacle.

Preferably, the first storage area is at least partially defined by an upper face, the first storage area being accessible through the upper face.

Preferably, in which the upper face comprises at least one opening providing access to a first interior region of the body which at least partially defines the first storage area.

Preferably, the second storage area comprises a plurality of discrete retention zones.

Preferably, the second storage area is at least partially defined by converging front and rear faces.

Preferably, the front and the rear faces together define a raised ridge in which the plurality of retention zones are formed.

Preferably, one or more of the retention zones comprise a slot formed in the body.

Preferably, the plurality of slots are arranged in a linear array.

Preferably, one or more of the slots tapers in a direction transverse to a longitudinal axis of the slot.

Preferably, the third storage area comprises an adhesion element to secure the one or more used sharps.

Preferably, the adhesion element comprises an adhesive.

Preferably, the adhesion element comprises an adhesive area surrounded on at least one side by a non-adhesive border.

Preferably, the adhesion element is divided into a plurality of adhesion zones.

Preferably, the third storage area is detachably secured to the body.

Preferably, the third storage area comprises a cover.

Preferably, the third storage area comprises a puncture resistant substrate.

Preferably, the body defines a base.

Preferably, the body is at least partially formed from recyclable material.

According to a second aspect of the present invention there is provided a blank for forming a storage system according to the first aspect of the invention.

As used herein, the term “surgical sharps” is intended to mean any surgical or medical equipment comprising one or more sharp edges, points or similar surfaces or features capable of piercing or cutting, and which is generally understood to mean suture needles, scalpel blades, syringe needles, etc.

### BRIEF DESCRIPTION OF THE DRAWINGS

The present invention will now be described with reference to the accompanying drawings, in which;

FIG. 1 a illustrates one face of a blank for forming a surgical sharps storage system according to a preferred embodiment of the present invention;

FIG. 1b illustrates an opposite face of the blank shown in FIG. 1 a;

FIG. 1c illustrates a cover locatable over a portion of the blank shown in FIGS. 1a and 1b;

FIG. 2 illustrates a perspective view of the blank of FIG. 1 folded to partially form the surgical sharps storage system of the invention;

FIG. 3 illustrates a perspective view of the surgical sharps storage system as illustrated in FIG. 2 but having a first storage area partially erected from panels of the blank;

FIG. 4 illustrates the arrangement of FIG. 3 with the first storage area fully erected;

FIG. 5 illustrates a perspective view of the surgical sharps storage system with a second storage area erected from panels of the blank;

FIG. 6 illustrates the surgical sharps storage system as shown in FIG. 5 and with a cover peeled back to expose a third storage area; and

FIG. 7 illustrates the surgical sharps storage system in which the third storage area has been separated from the rest of the system for disposal.

### DETAILED DESCRIPTION OF THE DRAWINGS

Referring now to the accompanying drawings there is illustrated a surgical sharps storage system, generally indicated as 10, for use in storing and accurately tracking the use of surgical sharps (not shown) such as but not limited to suture needles, scalpel blades, etc.

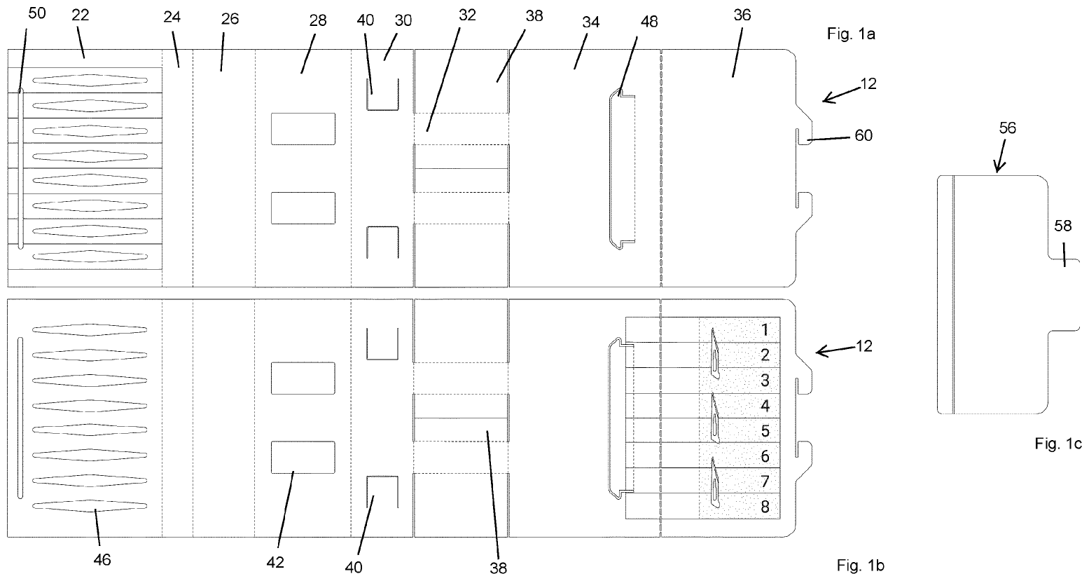

In the preferred embodiment illustrated the storage system 10 is folded from a planar blank 12 illustrated in FIGS. 1a and 1b and formed for example from one or more sheets of plastic, thick paper, card, cardboard or any other suitable material. The blank 12 may for example be formed from a laminate of different materials. It should however be understood from the following description of the configuration and operation of the storage system 10 that any other suitable method of manufacturing the storage system 10 may be employed, without requiring the use of the foldable blank 12. The use of a foldable blank 12 does however provide a number of advantages, both in significantly reducing the cost of the storage system 10, and allowing for an extremely compact form for transport and storage prior to use, and enabling the use of readily recyclable materials from which the blank 12 may be formed, for example plastic, cardboard or the like.

The two dimensional blank 12 of the storage system 10 is foldable as hereinafter described to define a three dimensional body 14 which comprises a number of defined sections, the body 14 essentially defining a first storage area 16 for one or more unopened packaged sharps (not shown), a second storage area 18 for one or more empty sharps packages (not shown), and a third storage area 20 for securing one or more used sharps (not shown).

In order to form the body 14 and the above storage areas 16, 18, 20, and referring primarily to FIGS. 1a and 1b, the blank 12 comprises first, second, third, fourth, fifth, sixth, seventh and eighth panels 22, 24, 26, 28, 30, 32, 34, 36 arranged in a linear array, adjacent panels being separated from one another by a respective fold line, or in the case of the seventh and eighth panel 34, 36 by a line of weakness in order to allow these panels to be separated from one another as described below. It will however be appreciated that the relative positions and connections between the various panels on the blank 12 may be varied once the panels can be folded together to form the body 14.

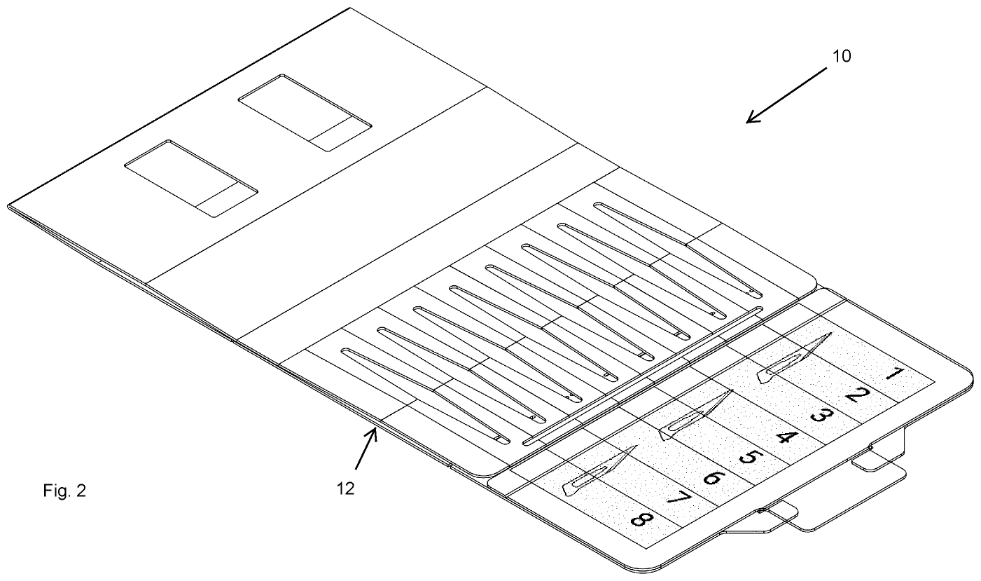

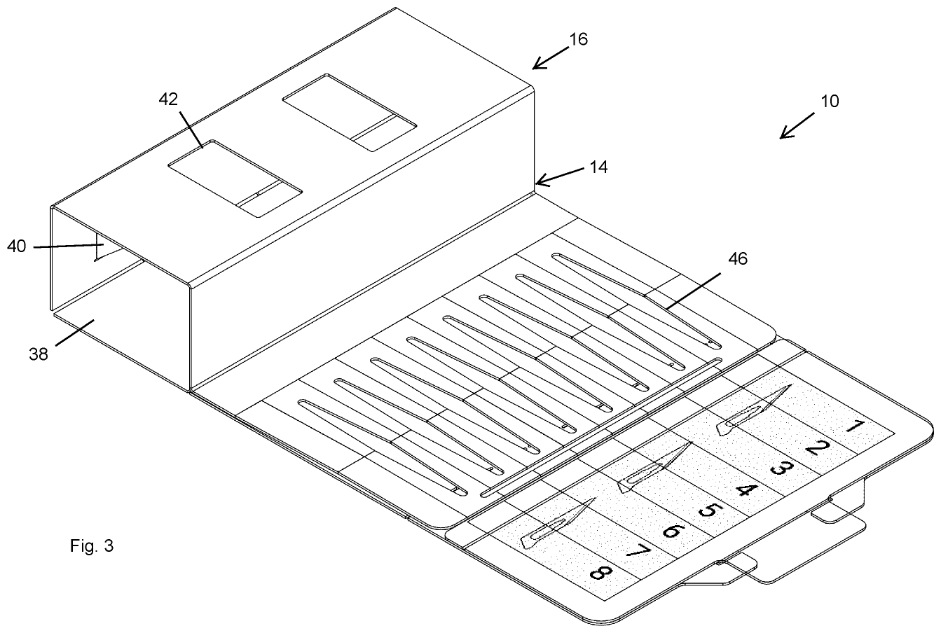

Referring to FIG. 2, in order to begin forming the storage system 10 from the blank 12, the blank 12 is folded over about the fold line dividing the fourth and fifth panels 28, 30, such that the first panel 22 overlies the seventh panel 34. Turning then to FIG. 3, the next step is to displace the fourth panel 28 upwardly away from the underlying sixth panel 32, which results in the third and fifth panels 26, 30 being drawn upright such that the panels 26, 28, 30, 32 form a box like enclosure defining the first storage area 16.

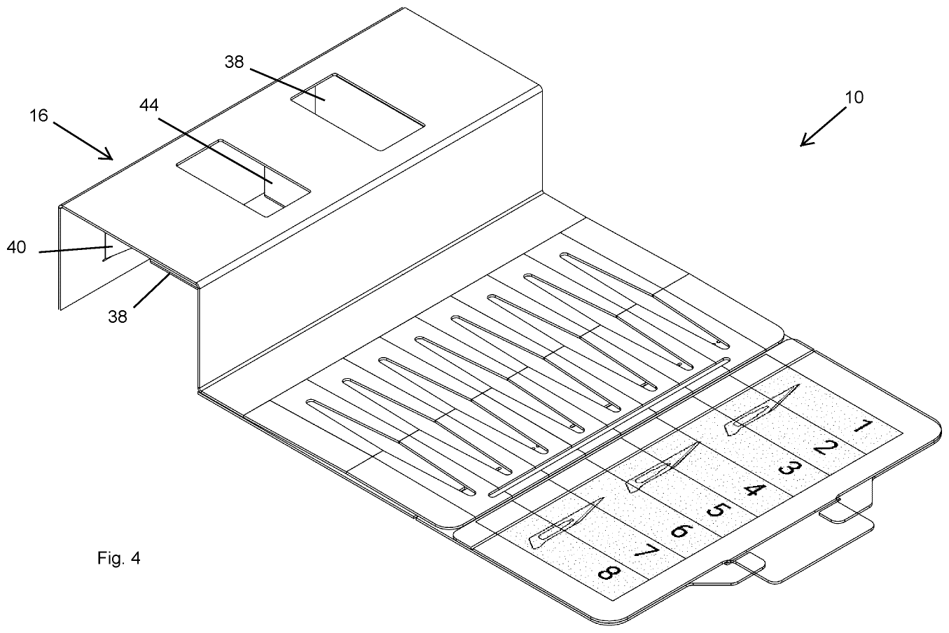

In order to maintain the shape of the storage area 16 and provide strength and structural integrity thereto, and referring to FIG. 4, a pair of internal walls 38, which are formed integrally with the sixth panel 32 by means of suitable cuts or lines of weakness in the sixth panel 32, are folded out of the sixth panel 32, one at either side therefore. The internal walls 38 are dimensioned to extend the full height between the fourth and sixth panel 28, 32 when positioned as shown in FIGS. 3 and 4.

FIG. 3 shows the internal walls 38 prior to being folded out from the sixth panel 32, while FIG. 4 shows the internal walls 38 following deployment. In order to secure the internal walls 38 in place a pair of tabs 40 are formed in the fifth panel 30, and are located such that when pressed out of the fifth panel 30 into the interior space defined by the first storage area 16 the tabs 40 act to prevent the internal walls 38 from returning back towards the plane of the sixth panel 32 under the inherent spring bias of the live hinge about which each internal wall 38 is folded.

The fourth panel 28 is provided with at least one, and in the embodiment illustrated, a pair of openings 42 formed therein, and which thus provide access to the interior space defined by the first storage area 16, thereby allowing packaged surgical sharps or the like to be located in the first storage area 16. The sixth panel 32 therefore preferably additionally defines a pair of dividing walls 44 which may again be folded inwardly into the interior space of the first storage area 16, and which are located to provide, along with the respective internal wall 38, a delineation of the interior space into two discrete volumes, one directly beneath each of the openings 42. Thus two separate storage sections are provided in the first storage area 16. The first storage area 16 is preferably dimensioned, in particular the depth of the interior space, such that packaged sharps stored therein will project partially out of the respective opening 42 in order to allow a medical professional to quickly access and select the appropriate sharp during a surgical procedure. The openings 42 should therefore also be appropriately dimensioned to hold the packaged sharps in an upright and ordered manner for ease of selection. It is also envisaged that multiple smaller openings may be provided which are dimensioned to receive a single sharps package therein, and in such an arrangement it is likely that six or eight such openings would be provided.

The first storage area 16 may also be adapted to actively engage the sharps packages to provide a positive hold thereon. For example the interior space could be provided with resiliently deformable material which would deform as a sharps package is inserted, and which would bear against the sharps package as a result of the resilience of the material, providing active retention of the sharps packages. Alternatively or additionally one or more edges of the openings 42 may be provided with a resiliently deformable wing projecting into the opening such that the wing will be deformed downwardly into the interior space as a packaged sharp is pressed into the opening, and the tendency of the wing to spring back will act to engage and thus grip the packaged sharp. In such an arrangement it is preferable to provide a pair of wings, one projecting from each of a pair of opposed edges of the opening.

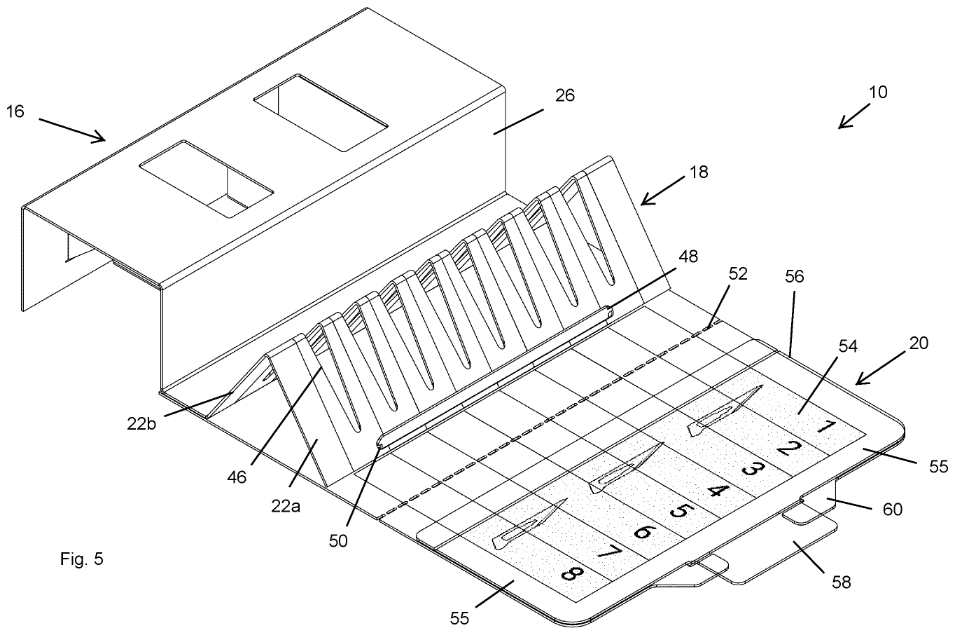

Turning now to FIG. 5 once the first storage area 16 has been formed from the blank 12, the second storage area 18 is formed by folding the first panel 22 in half to create a ridged or pitched structure between a front wall 22a and a rear wall 22b which defines the second storage area 18, and which is located adjacent the first storage area 16. The second storage area 18 further comprises an array of retention zones in the form of slots 46 cut out of the first panel 22 and dimensioned such that each slot 46 extends between the front and rear walls 22a, 22b and are thus open and accessible via the apex of the ridge formed by the walls 22a, 22b. The slots 46 are preferably arranged in parallel to one another, and may be provided in any appropriate number, eight slots 46 being provided in the preferred embodiment illustrated. Each slot 46 is shaped and dimensioned to receive and retain an empty sharps packet (not shown) therein, in order to ensure that the empty packages may be quickly and easily tracked and matched with used sharps at the end of the surgical procedure. The slots 46 preferably taper in a direction transverse to a longitudinal axis of the slot 46, narrowing in width in a direction downwardly into the slot 46 from the ridge or apex separating the front and rear walls 22a, 22b. In this way an empty package can be pressed downwardly into the slot 46 in order to effectively wedge the package into the slot 46 to ensure robust retention within the slot 46. It is also envisaged that an outer face of the third panel 26, which in use faces the second storage area 18, could be provided with openings or the like corresponding to the slots 46 and into which an end of a respective empty sharps package could also be inserted to supplement the retention provided by the slots 46. There could equally be an area of adhesive on the outer face of the third panel 26 to provide this functionality.

It will also be appreciated that any other suitable arrangement may be employed to provide the retention functionality of the second storage area 18. The particular arrangement of the font and rear walls 22a, 22b in forming the second storage area 18 is merely one of various configurations that could be employed to achieve the above mentioned retention functionality. For example the pitched configuration could be supplemented with a second pitched structure such as to define a substantially “M” shaped profile with each ridge having an array of slots formed therein. In this way the empty sharps packages would be held in two slots and extending between the pair of ridges. Alternatively the second storage area could comprise a curved wall, effectively defining a semi-cylindrical wall in which an array of slots could be defined to receive the empty sharps packages. Further alternatively the second storage area could be similar in form to the first storage area, providing a large enclosure into which the empty packages are simply deposited, although such an arrangement does not permit the empty packages to be quickly and easily counted.

In order to retain the front and rear walls 22a, 22b in the erected or raised position as illustrated for example in FIGS. 3 and 4, a locking tab 48 is formed integrally with and deformable out of the seventh panel 34, with a correspondingly shaped and dimensioned elongate slit 50 being cut out of the first panel 22 adjacent the lower or front edge of the front wall 22a, into which the tab 48 may be located in order to hold the first panel 22 in the erected configuration. The tab 48 is preferably barbed on either side as illustrated in order to prevent the unintended separation of the tab 48 and slit 50. It will of course be understood that any other functional alternative may be provided to the tab 48 and slit 50, for example an area of adhesive or the like provided on one or both of the first panel 22 and seventh panel 34, located at the overlap of the panels 22, 34 when the front and rear walls 22a, 22b are in the erected state. However the use of a reversible lock such as provided by the tab 48 and slit 50 between the panels 22, 34 is preferred, allowing the system 10 to be collapsed flat after use for disposal purposes.

Finally, the storage system 10 additionally comprises the third storage area 20 which projects forwardly of the second storage area 18, and is primarily defined by the eighth panel 36 which is separable from the seventh panel 34 by a line of weakness 52, which may for example comprise a row of perforations or the like in the material of the blank 12. The upper face of the eighth panel 36 comprises an adhesion element adapted to retain one or more used surgical sharps, and in the preferred embodiment illustrated comprises an area of adhesive on the face of the panel 36 onto which one or more surgical sharps can be placed and which will thus retain the surgical sharps thereon. The adhesion element is preferably visually and/or physically separated into a number of adhesion zones 54, which in the preferred embodiment illustrated are consecutively numbered rows or strips, and which may also be differentiated from one another by additional means such as colour coding or the like. The adhesion zones 54 are equal in number to the slots 46 so as to quickly and easily allow a user to match the number of empty packages retained in the slots 46, at the end of the surgical procedure, to the number of used sharps held in the third storage area 20.

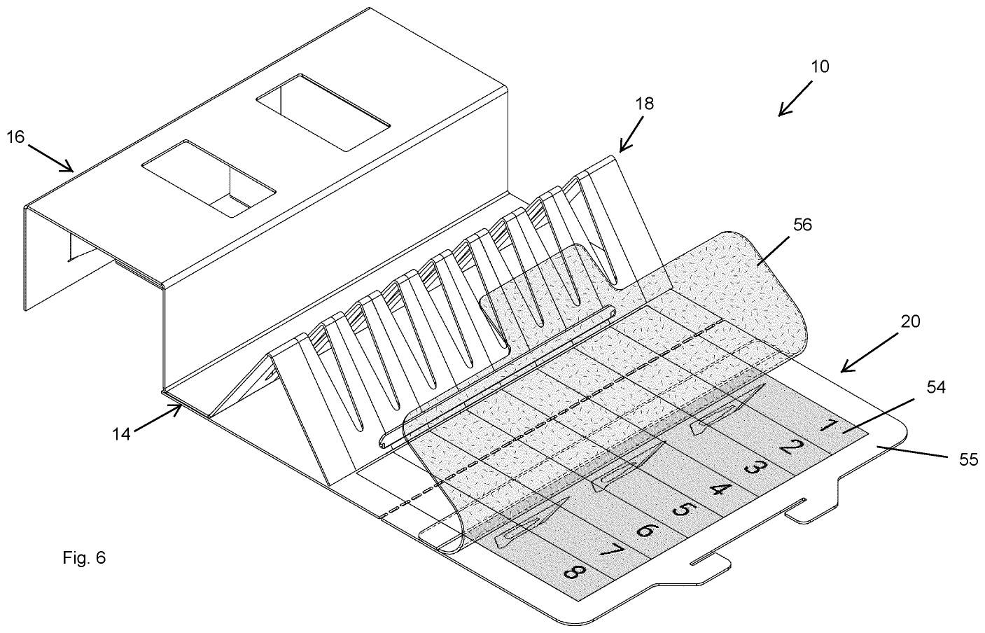

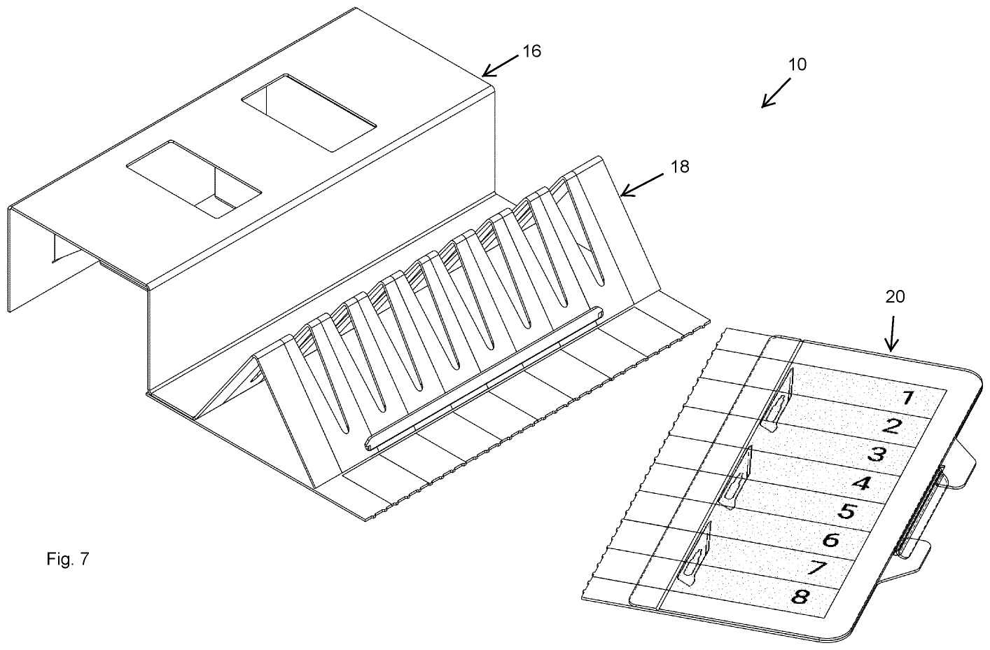

The adhesive area defined by the plurality of rows 54 is preferably surrounded on at least one side, and preferably all sides, by an non-adhesive border 55 which, during a surgical procedure, reduces the chance of swabs or the like sticking to the third storage area 20 and allows constant visibility of used sharps. In order to further reduce such an occurrence, the third storage area 20 preferably comprises a cover 56 which in the embodiment illustrated is in the form of a transparent sheet of material, for example BoPET (Mylar) or other suitable polymer, which can be at least partially peeled back out of contact with the panel 36 to permit access to the adhesion zones 54 when a used surgical sharp is to be deposited thereon, as illustrated in FIG. 6. The cover 56 is shown in isolation in FIG. 1c, and in use is adhered to the eighth panel 36 along a rear edge of the cover 56 to fully overlie the adhesion zones 54. The underside of the cover 56 may be provided with a non stick coating or layer, such as a silicone based release layer, in order to prevent the cover 56 from sticking to the adhesion zones 54. The cover 56 comprises a tab 58 at the front edge thereof, and the eighth panel 36 is provided with a corresponding receiver 60 into which the tab 58 may be reversibly inserted in order to hold the cover 56 in the closed position. This is preferable when the surgical procedure is complete and the used surgical sharps are to be disposed of. It is also preferable that the cover 56 is transparent to allow the used surgical sharps to be counted with the cover 56 in place. However any other suitable form of cover may be provided.

The substrate forming at least the portion of the blank 12 that defines the third storage area 20, and in the preferred embodiment illustrated the eighth panel 34, is preferably formed of puncture resistant material in order to prevent the used surgical sharps from piercing the panel 34, which could lead to user contact with the used sharps during disposal of the third storage area 20.

It should be understood from the above that the adhesion element does not functionally require the use of an adhesive, and is merely intended to mean an element capable of holding or retaining one or more surgical sharps, and could for example comprise a portion of foam or gel into which a surgical sharp such as a needle may be inserted for retention, for example in the manner of a so called pin cushion. Further alternatively a magnetic material could be employed if the surgical sharps to be retained comprise ferrous material.

Thus in use the blank 12 is folded as described above in order to erect the storage system into the configuration shown in FIGS. 5 and 6. The underside of the system 10 when fully erected, for example on the seventh panel 34, may be provided with one or more non slip feet or the like, which could be defined by one or more patches or areas of adhesive, in order to allow the system 10 to be securely fixed to an appropriate working surface. The surgeon or other member of the medical team will then procure the full set of medical sharps (not shown) to be used during a given medical procedure. For example the procedure may require the use of six suture needles, each of which is individually sealed in a sterile package which conventionally takes the form of a small sleeve like enclosure. The openings 42 are shaped and dimensioned to each allow a number of such packages to be inserted therein, but as mentioned above the openings 42 could be replaced with a larger number, for example eight, dedicated slots or openings into which an individual one of said sealed packages can be at least partially inserted. The openings 42 in the preferred embodiment are shaped and dimensioned to permit multiples of such sterile packages, stacked together, to be partially inserted into the first storage area 16, thereby projecting partially out of the openings 42 which will then serve to retain the unopened sharps packages while being externally accessible to allow a surgeon or other member of the medical team to retrieve one of the sharps packages as required.

During the surgical procedure and as required an unopened surgical sharp can be removed from the first storage area 16 and the suture needle or the like removed from the package for use. The empty package is immediately inserted into one of the slots 46 in the second storage area 18, and pressed downward to ensure secure retention as hereinbefore described.

Once the suture needle or other surgical sharp has been used it may then be placed onto one of the adhesion zones 54, most preferably the zone 54 corresponding to the slot 46 into which the empty package was placed. The cover 56 is peeled back as shown in FIG. 6 in order to allow the used sharp to be deposited.

The above procedure is repeated until the surgery is complete, following which there should be a plurality of empty packages secured with a number of the slots 46, and a corresponding number of needles or other surgical sharps adhered or otherwise secured in the third storage area 20. The packages and used sharps can then be quickly and easily counted to ensure they match, thereby ensuring that no needles have been lost or inadvertently left internally of the patient.

At this point the cover 56 is locked in place using the tab 58 and receiver 60, and the third storage area 20 may then be separated from the body 14, by tearing the eighth panel 36 from the seventh panel 34 via the line of weakness 52. If required a second count of the used sharps may be taken at this time, which can be achieved with the cover 56 in place due to the transparency thereof. The separated third storage area 20, containing the used sharps, can then be disposed of in the appropriate manner. The empty suture packs can be removed from the slots 46 and discarded in a separate container. The body 14 of the storage system 10 may then collapsed flat and discarded, preferably for recycling.

It will thus be appreciated that the storage system 10 of the present invention provides a simple and cost effective means of dispensing and tracking the use of surgical shapes, and which allows the used sharps to be disposed in a conventional sharps bucket but will take up an extremely small amount of space and will result in significant cost savings for disposal.

1. A surgical sharps storage system comprising a body defining a first storage area for one or more packaged sharps; a second storage area for one or more empty sharps packages; and a third storage area for securing one or more used sharps.

2. The surgical sharps storage system of claim 1, wherein the body is formed from at least one planar blank comprising a plurality of panels foldable to form at least one from the group consisting of the first, the second, and the third storage areas.

3. The surgical sharps storage system of claim 1, wherein the first storage area comprises at least one receptacle.

4. The surgical sharps storage system of claim 3, wherein the first storage area is at least partially defined by an upper face, the first storage area being accessible through the upper face.

5. The surgical sharps storage system of claim 4, wherein the upper face comprises at least one opening providing access to a first interior region of the body which at least partially defines the first storage area.

6. The surgical sharps storage system according to of claim 1, wherein the second storage area comprises a plurality of discrete retention zones.

7. The surgical sharps storage system of claim 6, wherein the second storage area is at least partially defined by converging front and rear faces.

8. The surgical sharps storage system of claim 7 wherein, the front and the rear faces together define a raised ridge in which the plurality of retention zones are formed.

9. The surgical sharps storage system of claim 6, wherein one or more of the retention zones comprise a slot formed in the body.

10. The surgical sharps storage system of claim 9, wherein the plurality of slots are arranged in a linear array.

11. The surgical sharps storage system of claim 10, wherein one or more of the slots tapers in a direction transverse to a longitudinal axis of the slot.

12. The surgical sharps storage system of claim 1, wherein the third storage area comprises an adhesion element to secure the one or more used sharps.

13. The surgical sharps storage system of claim 12, wherein the adhesion element comprises an adhesive.

14. The surgical sharps storage system of claim 13, wherein the adhesion element comprises an adhesive area surrounded on at least one side by a non-adhesive border.

15. The surgical sharps storage system of claim 13, wherein the adhesion element is divided into a plurality of adhesion zones.

16. The surgical sharps storage system of claim 1, wherein the third storage area is detachably secured to the body.

17. The surgical sharps storage system of claim 1, wherein the third storage area comprises a cover.

18. The surgical sharps storage system of claim 1, wherein the third storage area comprises a puncture resistant substrate.

19. The surgical sharps storage system of claim 1, wherein the body defines a base.

20. The surgical sharps storage system of claim 1, wherein the body is at least partially formed from recyclable material.

21. The blank for forming a storage system according to claim 1.
